(19) United States
(12) Patent Application Publication
(10) Pub. No.: US 2025/0017817 A1
(43) Pub. Date: Jan. 16, 2025

(54) ACOUSTIC SHOCK WAVE TREATMENT AND DEVICES FOR EXCESS, SAGGING SKIN

(71) Applicant: John F. Warlick, Kennesaw, GA (US)

(72) Inventor: John F. Warlick, Kennesaw, GA (US)

(21) Appl. No.: 18/219,881

(22) Filed: Jul. 10, 2023

(51) Int. Cl. A61H 23/04; A61H 23/02; A61M 37/00

(52) U.S. Cl. CPC A61H 23/04; A61H 23/0245; A61M 37/0092; A61H 2201/0153; A61H 2201/0207; A61H 2201/105; A61H 2201/5005; A61M 2037/0007

(57) ABSTRACT

An improved method of treating excess, sagging skin of a patient using acoustic shock waves has the steps of: providing a patient with excess sagging skin tissue in need of an acoustic shock wave treatment; applying a liquid based topical treatment to surfaces of the excess sagging skin and an acoustic shock wave applicator to acoustically couple skin surfaces to enhance transmission of the acoustic shock waves; placing an acoustic shock wave applicator on a surface of the skin tissue; and activating an acoustic shock wave generator or source to emit acoustic shock waves from an acoustic shock wave applicator.

### FIELD OF THE INVENTION

The present invention relates to improved treatments of excess, sagging skin for repairing or regenerating dermal cells, tissues or vessels to overcome various degenerative skin conditions, more particularly sagging skin.

### BACKGROUND OF THE INVENTION

The use of treating skin using acoustic shock waves is known by the inventors of the present invention to treat various conditions such as poor circulation, arthritis in the hands or feet, organs or other body parts. People with obesity may lose weight with bariatric weight loss surgery or diet and exercise. Significant rapid weight loss can result in excess, sagging skin. When extra weight is gained, skin and tissue stretch to accommodate the body's larger size. This stretching causes skin and tissue to lose elasticity. When this extra weight is lost, the stretched skin can't conform easily and may be saggy and hang loosely. Having loose skin can result in physical discomfort, self-consciousness, decrease in physical activity, skin irritation or breakdown and poor body image.

When skin has been significantly stretched and remains that way for a long period of time, collagen and elastin fibers become damaged. As a result, they can lose some of their ability to retract. Also, it has been found that some patients form less new collagen, and its composition is not as strong as the collagen in young, healthy skin.

Collagen hydrolysate is very similar to gelatin. It's a processed form of the collagen found in the connective tissue of animals. Although it hasn't been tested in people with loose skin related to major weight loss, studies suggest that collagen hydrolysate may have a protective effect on skin's collagen. In a controlled study, skin elasticity, along with texture and hydration, increased significantly after 12 weeks of taking a liquid collagen supplement. The drink also contained vitamins C and E, as well as biotin and zinc. Collagen hydrolysate is also known as hydrolyzed collagen. It comes in powdered form and can be purchased at natural food stores or online. Another popular source of collagen is bone broth, which provides other health benefits as well.

Certain nutrients are important for the production of collagen and other components of healthy skin: Protein. Adequate protein is vital for healthy skin. The amino acids lysine and proline play a direct role in collagen production. Vitamin C. Vitamin C is needed for collagen synthesis and also helps protect skin from sun damage.

Omega-3 fatty acids. The omega-3 fatty acids found in fatty fish may help increase skin elasticity and have anti-aging effects. Water. Staying well hydrated may improve the skin's appearance. One study found that women who increased their daily water intake had significant improvements in skin hydration and function.

Many “firming” creams contain collagen and elastin. Although these creams may temporarily give a slight boost to skin tightness, collagen and elastin molecules are too large to be absorbed through your skin. In general, collagen must be created from the inside out.

Body-contouring surgery is the most common procedure to remove loose skin, less invasive options with a lower risk of complications include: Radiofrequency treatment. Such treatments may use a combination of infrared light, radiofrequency, and massage to reduce loose skin. While this treatment doesn't induce weight loss, it may help reduce small areas of fat cells. Ultrasound. A controlled study of ultrasound treatment in people who had bariatric surgery found no objective improvement in loose skin. However, people did report relief of pain and other symptoms following treatment. It appears that although there are fewer risks with these alternative procedures, the results may not be as dramatic as with body-contouring surgery.

The present invention has found a way to more effectively treat this excess skin with faster and more reliable results by applying acoustic shock waves directly to the excess skin with an acoustic shock wave treatment for excess/loose skin after a substantial weight loss. This, when combined with a liquid based topical treatment that couples the skin acoustically to the shock wave applicator, enhances the shock wave treatment and drives the topical treatment deep into the dermis tissue.

These and other objectives are achieved using the inventive technology described herein. The present invention provides new improved skin treatment methods and devices to accomplish these improvements as described hereinafter.

### SUMMARY OF THE INVENTION

An improved method of treating excess, sagging skin of a patient using acoustic shock waves has the steps of: providing a patient with excess sagging skin tissue in need of an acoustic shock wave treatment; applying a liquid based topical treatment to surfaces of the excess sagging skin and an acoustic shock wave applicator to acoustically couple skin surfaces to enhance transmission of the acoustic shock waves; placing an acoustic shock wave applicator on a surface of the skin tissue; activating an acoustic shock wave generator or source to emit acoustic shock waves from an acoustic shock wave applicator; and wherein the acoustic shock waves are transmitted from the acoustic shock wave applicator through the surface sending the emitted acoustic shock waves into the skin tissue of the patient by holding or pressing the applicator firmly against skin surfaces to enhance the acoustic coupling and to drive a portion of the topical treatment below the skin surface into the dermis tissue.

The step of activating the acoustic shock wave generator or source emits low energy or unfocused acoustic shock waves. The acoustic shock waves are waves having amplitudes above 0.1 MPa and rise times of the amplitude are below 100 nano-seconds with a duration of a shock wave being below 3 micro-seconds for the positive part of a cycle and wherein the pressure pulses are an acoustic pulse which includes several cycles of positive and negative pressure with amplitudes of the positive part of such a cycle being above 0.1 MPa and the pressure pulse time duration is from below a microsecond to about a second, rise times of the positive part of the first pressure cycle is in the range of nano-seconds up to several milli-seconds.

The method of treating excess sagging skin of a patient using acoustic shock waves further includes subjecting the skin to the acoustic shock waves stimulating said excess sagging skin, the skin is positioned within a path of the emitted shock waves stimulating a cellular response.

In a preferred embodiment, the acoustic shock wave applicator is electrohydraulic and has a fluid filled flexible membrane. Other types of applicators including ballistic, piezoelectric, radial or spherical applicator devices can be used.

The emitted shock waves or pressure pulses are convergent, divergent, planar or near planar. The emitted shock waves or pressure pulses are convergent having one or more geometric focal volumes or points located at a distance X, X being defined as the distance from an exit window to the one or more focal volumes or points from the generator or source, the excess sagging skin being positioned at the distance X or less than the distance X from the exit window source.

The method of treatment further has the step of increasing the temperature of the skin being treated to change tissue impedance to improve tissue stimulation by opening pores of the skin to absorb the topical treatment deep into the dermis below the surface as the acoustic shock waves impinge the skin.

### Definitions

“Collagen”: Collagen essentially acts as the support structure—or scaffolding—for the skin. Collagen is the most abundant protein in the body and makes up around 70-80% of the dry weight of skin, according to the US National Library of Medicine/National Institutes of Health. As collagen production dips, the connective tissue between the skin becomes thinner and the layer of fat underneath it becomes more visible, leading to cellulite on the body. On the face, skin can become thinner and more fragile, and wrinkles may be more visible. According to the Merck Manual, the dermis, the layer of skin below the epidermis, is a thick layer of fibrous and elastic tissue, made mostly of collagen, with a small but important component of elastin that gives the skin its flexibility and strength. A form of collagen therapy is injecting collagen directly into the dermis. When injected into the body's skin both forms of collagen are accepted as if they were the body's collagen, forming a network of collagen fibers.

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“Elastin” is what gives skin its “bounce back” property, as the name implies. According to the University of Leeds, elastin is what makes tissue recoil after being stretched, just like a rubber band-but elastic fibers are five times more extensible than an elastic band. Like collagen, elastin is a protein that is found in the body's connective tissue. It is found in the skin as well as the lungs, intestines and artery walls. As elastin levels decline, skin can wrinkle and sag.

“extracorporeal” occurring or based outside the living body.

A “generalized paraboloid” according to the present invention is also a three-dimensional bowl. In two dimensions (in Cartesian coordinates, x and y) the formula yⁿ=2px [with n being ≠2, but being greater than about 1.2 and smaller than 2, or greater than 2 but smaller than about 2.8]. In a generalized paraboloid, the characteristics of the wave fronts created by electrodes located within the generalized paraboloid may be corrected by the selection of (p (−z,+z)), with z being a measure for the burn down of an electrode, and n, so that phenomena including, but not limited to, burn down of the tip of an electrode (−z,+z) and/or disturbances caused by diffraction at the aperture of the paraboloid are compensated for.

“Glycerin”: Natural glycerin is found in animal and plant fats. Similarly, when fats and oils are hydrolyzed to yield fatty acids or soaps, glycerin is formed. Glycerin is a solvent, food additive, sweetening agent and emollient as well as a demulcent, an agent that forms a soothing film. Glycerin can be used to treat septic wounds and boils, a component of antifreeze, in the manufacture of resins and cellophane, ester gums, plasticizers, dynamite, nitroglycerine, cosmetics, liquid soap, perfume and toothpaste. It also helps keep fabrics pliable, preserves the printing on cotton, and prevents frost from forming on windshields. In research, glycerin is often used as the source of nutrients for fermentation cultures and can act as a preservative as well.

A “paraboloid” according to the present invention is a three-dimensional reflecting bowl. In two dimensions (in Cartesian coordinates, x and y) the formula y²=2px, wherein p/2 is the distance of the focal point of the paraboloid from its apex, defines the paraboloid. Rotation of the two-dimensional figure defined by this formula around its longitudinal axis generates a de facto paraboloid.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nano-seconds (ns) up to some milli-seconds (ms). Very fast pressure pulses are called shock waves. Shock waves used in medical applications do have amplitudes above 0.1 MPa and rise times of the amplitude can be below 1000 ns, preferably at or below 100 ns. The duration of a shock wave is typically below 1-3 micro-seconds (μs) for the positive part of a cycle and typically above some micro-seconds for the negative part of a cycle.

“Shock Wave”: As used herein is defined by Camilo Perez, Hong Chen, and Thomas J. Matula; Center for Industrial and Medical Ultrasound, Applied Physics Laboratory, University of Washington, 1013 NE 40th Street, Seattle, Washington 98105; Maria Karzova and Vera A. Khokhlovab; Department of Acoustics, Faculty of Physics, Moscow State University, Moscow 119991, Russia; (Received 9 Oct. 2012; revised 16 Apr. 2013; accepted 1 May 2013) in their publication, “Acoustic field characterization of the Duolith: Measurements and modeling of a clinical shock wave therapy device”; incorporated by reference herein in its entirety.

Wave energy or energy flux density: the measurement of energy flux density is defined as the energy directed toward the target or region being treated. This is not energy at the gap between electrodes, but rather the energy transmitted toward the patient's tissue through the skin. Important to distinguish that the energy levels discussed pertain to the energy delivered to the targeted tissues and not at the discharge point between the electrode tips. Spherical waves have a huge amount of energy produced between the tips to deliver adequate energy to the targeted tissues since they do not have the advantage of a lens.

“Coupling gel, acoustic/ultrasound”: The primary ingredient in ultrasound gel is water, which is used to create the gel-like consistency that allows for easy application and minimal friction between the transducer and the patient's skin. Additionally, water has a similar acoustic impedance as the skin, which means that it allows the sound waves to pass through with minimal reflection. Another important ingredient in ultrasound gel is a thickener or gelling agent. This ingredient is used to create the gel-like consistency and to ensure that the gel remains in place during the procedure. Common thickeners used in ultrasound gel include carbomers and xanthan gum. Another ingredient used in ultrasound gel is a preservative. Preservatives are used to prevent the growth of microorganisms in the gel, ensuring that it remains safe for use. Common preservatives used in ultrasound gel include parabens and benzyl alcohol. Some gels also contain a humectant, which is an ingredient that helps to prevent the gel from drying out. Humectants such as glycerin and propylene glycol are commonly used in ultrasound gel. Another ingredient that can be found in some ultrasound gels is a lubricant, which improves the patient's comfort during the procedure. Lubricants such as mineral oil and glycerin are commonly used in ultrasound gel.

Waves/wave fronts described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

### BRIEF DESCRIPTION OF THE DRAWINGS

The invention will be described by way of example and with reference to the accompanying drawings in which:

FIG. 1 is a simplified depiction of a pressure pulse/shock wave (PP/SW) generator with focusing wave characteristics.

FIG. 2 is a simplified depiction of a pressure pulse/shock wave generator with plane wave characteristics.

FIG. 3 is a simplified depiction of a pressure pulse/shock wave generator with divergent wave characteristics.

FIG. 4 is a perspective view of a shock wave generator device.

FIG. 5 is a graph showing an exemplary ultrasound wave pattern.

FIG. 6 is a graph of an exemplary acoustic shock wave pattern.

FIG. 7 is a perspective view of a patient being treated with acoustic shock waves.

### DETAILED DESCRIPTION OF THE INVENTION

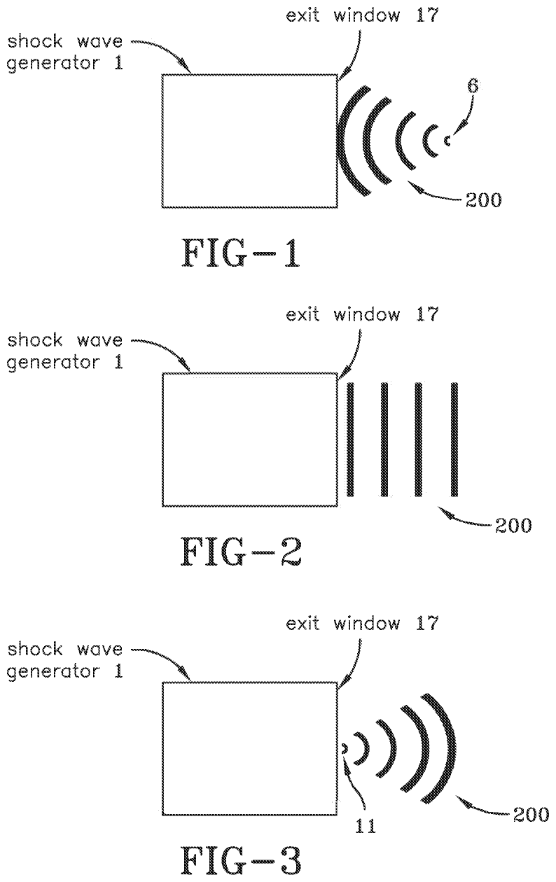

With reference to the figures, FIGS. 1-3 show various shock waves 200 being transmitted from a shock wave generator 1. FIG. 1 is a focused shock wave wherein the shock wave pattern emits from an exit window 17 and focuses to a point or localized area 6 as illustrated. The wave form 200 is considered a converging wave form. In FIG. 2, the shock wave emits from the exit window 17 in a planar fashion wherein the wave forms 200 are transmitted parallel to the exit window 17 and transmitted into the patient. In FIG. 3, the wave form 200 is emitted from the exit window 17 in such a fashion that the focal point is near the exit window 17 and the wave form 200 expands outwardly, this is considered a divergent wave form wherein the wave form expands as it leaves the exit window 17 in a diverging pattern.

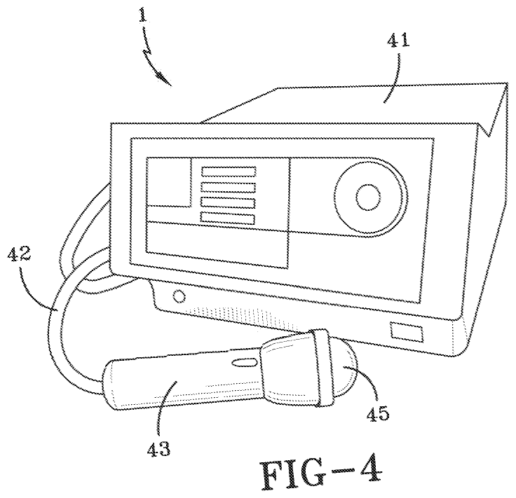

FIG. 4 shows an exemplary shock wave device generator or source 1 with a control and power supply 41 connected to a hand-held applicator shock wave head 43 via a flexible hose 42 with fluid conduits. The illustrated shock wave applicator 43 has a flexible membrane 45 at an end of the applicator 43 which transmits the acoustic waves when coupled to the external surface of excess or sagging skin of dermal tissue of a patient by using a fluid topical treatment or acoustic gel. The topical treatment can be used without any incidence of infection as the acoustic shock waves produce a natural germicidal effect. As shown, this type of applicator 43 has a hydraulic spark generator using either focused or unfocused shock waves, preferably in a low energy level, less than the range of 0.01 mJ/mm² to 0.3 mJ/mm². The flexible hose 42 is connected to a fluid supply that fills the applicator 43 and expands the flexible membrane 45 when filled. Alternatively, a ballistic, piezoelectric or spherical acoustic shock wave device can be used to generate the desired waves. The fluid expands a flexible membrane 45 in such a fashion that the membrane 45 extends outwardly in a balloon shape fashion as illustrated in FIG. 4.

The topical treatment can contain ingredients such as collagen, glycerin, elastin, live yeast cell derivative, hyaluronic acid, preservatives, protein fractions, peptides and other suitable skin treatment components.

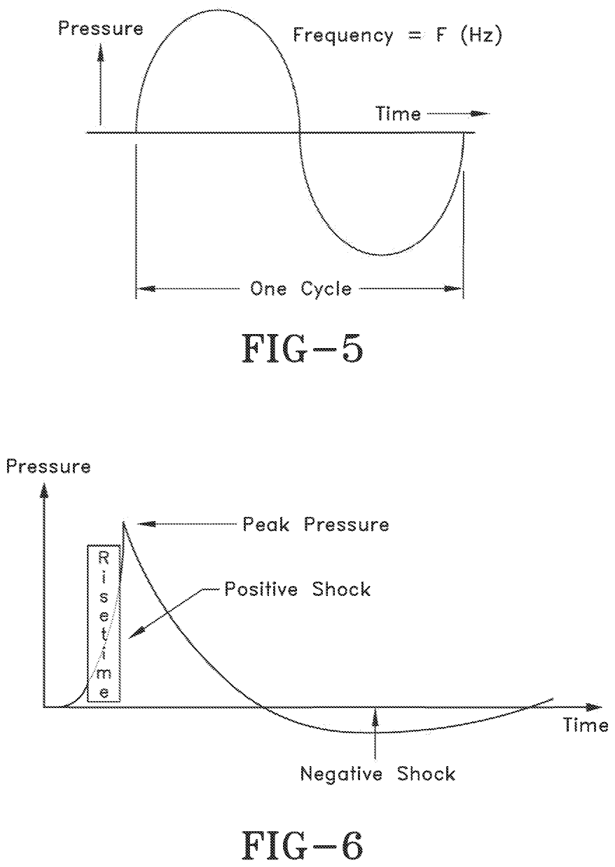

An important aspect of the present invention is that the pressure waves from the acoustic shock wave generator have an asymmetric type wave form with a very high peak pressure that occurs over a very short rise time. The positive shock is transmitted in a very quick fashion as defined which defines the features of an acoustic shock wave or pressure pulse. The negative portion of the wave is longer in duration and encompasses the rest of the wave form as shown in FIG. 6. This is unlike ultrasound wave forms which are symmetrical, sinusoidal in shape as illustrated in FIG. 5. The main difference between an acoustic shock wave and an ultrasound wave is that there is no heat generated in the asymmetric type acoustic shock wave whereas there is heat generation in the ultrasound wave. The ultrasound wave therefore is generally considered inferior for the purposes of tissue treatments compared to the use of the electrohydraulic acoustic shock waves emitted from the applicator 43. The applicator 43 as shown is electrohydraulic, but it could be ballistic, piezoelectric or any other form of applicator exhibiting the asymmetrical waves. The asymmetric acoustic wave pattern shown in FIG. 6 is contrasted to an ultrasonic wave pattern which is illustrated in FIG. 5. As shown, ultrasound waves are symmetrical having the positive rise time equal to the negative in a sinusoidal wave form. These ultrasound waves generate heat in the tissue and are accordingly believed not as suitable for use on tissue requiring a cellular stimulation to achieve the desired goals of the treatment method.

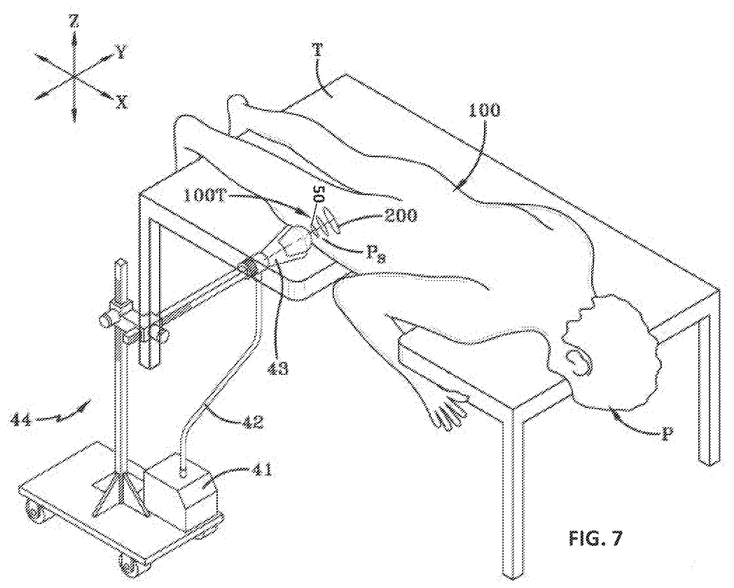

With reference to FIG. 7, the patient P who has excess sagging skin is positioned on a table T. A shock wave applicator head 43 is brought into contact with the skin Ps of a leg 100T preferably an acoustic gel 50 is used to enhance the transmission of the shock waves 200 through the body down to the subsurface extremity 100 containing a portion of the vascular system 101 surrounded by muscle tissue 102. The shock wave applicator head 43 is connected via cabling 42 to a power generating unit 41 as shown. The shock wave applicator head 43 can be attached rigidly to a fixture or stand 44 as illustrated or alternatively can be handheld and manipulated across the skin Ps to drive the shock waves 200 in the direction the shock wave head 43 is pointed.

In the extracorporeal shock wave or pressure pulse method of treating tissue, the administered shock waves or pressure pulses are directed to a treatment location or target site on the tissue. As used herein, “near” recognizes that the emitted shock waves or pressure pulses are transmitted through the tissue, preferably at or in close proximity to the treatment location or site.

Assuming the target area is within a projected area of the wave transmission, a single transmission dosage of wave energy may be used. The transmission dosage can be from a few seconds to 20 minutes or more dependent on the condition. Preferably the waves are generated from an unfocused or focused source. The unfocused waves can be divergent or near planar and having a low-pressure amplitude and density in the range of 0.00001 mJ/mm² to 1.0 mJ/mm² or less, most typically below 0.2 mJ/mm². These are typically generated by spherical or radial wave generators, ballistic or electrohydraulic wave or piezoelectric shock wave generators. The focused source can use a focused beam of waves or can optionally use a diffusing lens or have a far-sight focus to minimize if not eliminate having the localized focus zone within the tissue. Preferably the focused shock waves are used at a similarly effective low energy transmission or alternatively can be at higher energy but wherein the tissue target site is disposed pre-convergence inward of the geometric focal point of the emitted wave transmission. Understanding the higher the energy used, the more sensation of bruising the tissue. In these cases, cavitation can and often does occur as well as bruising and come cell damage. This is preferably and easily avoidable by employing the low energy as previously discussed.

These shock wave energy transmissions are effective in stimulating a cellular response and in some cases, such as unfocused low energy, and even low energy focused emissions can be accomplished without creating the localized hemorrhaging caused by rupturing cavitation bubbles in the tissue of the target site. This effectively ensures the tissue does not have to experience the sensation of cellular damage so common in the higher energy focused wave forms having a focal point at or within the targeted treatment site. Higher energy acoustic shock waves or pressure pulses including focused waves can be used, but with care to avoid such damage.

The target site may be such that the tissue or the generating source must be reoriented relative to the site and a second, third or more treatment dosage can be administered. At a low energy, the common problem of localized hemorrhaging is reduced making it more practical to administer multiple dosages of waves from various orientations to further optimize the treatment and cellular stimulation of the target site. Alternatively, focused high energy multiple treatments can be equally effective, but with some risk to tissue bruising. The use of low energy focused or un-focused waves at the target site enables multiple sequential treatments. Alternatively, the wave source generators may be deployed in an array wherein the subject tissue is effectively enveloped or surrounded by a plurality of low energy wave source generators which can be simultaneously bombarding the target site from multiple directions. Such arrays include linear type devices.

The goal in such treatments is to provide 100 to 3000 acoustic shock waves or pressure pulses. Typically, at a voltage of 14 kV to 28 kV across a spark gap generator in a single treatment preferably or one or more adjuvant treatments by targeting the site directly by impinging the emitted waves toward the regions of excessive sagging skin.

The present method does not rely on precise site location per se. The physician's general understanding of the anatomy should be sufficient to locate a desirable direct path or to the target site to attack the condition being treated. The treated area can withstand a far greater number of shock waves based on the selected energy level being emitted. For example, at very low energy levels the stimulation exposure can be provided over prolonged periods as much as 20 minutes if so desired. At higher energy levels the treatment duration can be shortened to less than a minute, less than a second if so desired. The selected treatment dosage can include the avoidance or minimization of cell hemorrhaging and other kinds of damage to the cells or tissue while still providing a stimulating cellular release activation of upregulation of the antimicrobial peptide LL37, a protein that can bind with RNA to destroy any infections, and also VEGF and other growth factors.

The underlying principle of these sound wave therapy methods is to stimulate the dermal tissue. This is accomplished by deploying shock waves to stimulate strong cells in the dermal tissue to activate a variety of responses, more particularly those that reduce inflammation and stop any infections. The sound waves including acoustic shock waves or pressure pulses transmit or trigger what appears to be a cellular communication throughout the entire anatomical structure of the dermal tissue, this activates a generalized cellular response at the treatment or target site, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced but also the number of applied shock wave impulses can be lowered from several thousand to as few as one or more pulses and still yield a beneficial stimulating response if desired.

The biological model motivated the design of sources with low pressure amplitudes and energy densities. First: spherical waves generated between two tips of an electrode; and second: nearly even waves generated by generated by generalized parabolic reflectors. Third: divergent shock front characteristics are generated by an ellipsoid. Unfocused sources are preferably designed for extended two dimensional areas/volumes like skin. The unfocused sources can provide a divergent wave pattern or a nearly planar wave pattern and can be used in isolation or in combination with focused wave patterns yielding to an improved therapeutic treatment capability that is non-invasive with few if any disadvantageous contraindications. Alternatively, a focused wave emitting treatment may be used wherein the focal point extends to the target site. In any event, the beam of acoustic waves transmitted needs to project in a large enough zone or area to stimulate the cells in the dermal tissue.

In one embodiment, the method of treatment has the steps of, generating either focused shock waves or unfocused shock waves, of directing these shock waves to the treatment site; and applying a sufficient number of these shock waves to induce activation of one or more growth factor or anti-microbial peptides like LL37, thereby inducing or accelerating a modulated adjustment to induce the host cells of the dermal tissue to attack any infection or disease.

The shock waves can be of a low peak pressure amplitude and density. Typically, the energy density values range as low as 0.000001 mJ/mm² and having a high end energy density of below 1.0 mJ/mm², preferably 0.40 mJ/mm² or less, more preferably 0.20 mJ/mm² or less. The peak pressure amplitude of the positive part of the cycle should be above 1.0 and its duration is below 1-3 microseconds.

The treatment depth can vary from the surface to the full depth of the dermal tissue and the treatment site can be defined by a much larger treatment area. The above methodology is particularly well suited for surface as well as sub-surface soft tissue treatments in dermal tissue.

An exemplary treatment protocol could have emitted shock waves in a broad range of 0.01 mJ/mm² to 3.0 mJ/mm² and 200-2500 pulses per treatment with a treatment schedule of 1-3 treatments being repeated over several weeks.

In the shock wave method of treating the excess sagging skin tissue of a patient requires the patient to be positioned in a convenient orientation to permit the source of the emitted waves to most directly send the waves to the target site to initiate shock wave stimulation of the target area with minimal, preferably no obstructing features in the path of the emitting source or lens. Assuming the target area is within a projected area of the wave transmission, a single transmission dosage of wave energy may be used. The transmission dosage can be from a few seconds to 20 minutes or more dependent on the condition. Preferably the waves are generated from an unfocused or focused source. The unfocused waves can be divergent, planar or near planar and having a low pressure amplitude and density in the range of 0.00001 mJ/mm² to 1.0 mJ/mm² or less, most typically below 0.2 mJ/mm². The focused source preferably can use a diffusing lens or have a far-sight focus to minimize if not eliminate having the localized focus point within the tissue. Preferably the focused shock waves are used at a similarly effective low energy transmission or alternatively can be at higher energy but wherein the tissue target site is disposed pre-convergence inward of the geometric focal point of the emitted wave transmission.

These shock wave energy transmissions are effective in stimulating a cellular response and can be accomplished without creating the cavitation bubbles in the tissue of the target site. This effectively insures the dermal tissue does not have to experience the sensation of hemorrhaging so common in the higher energy focused wave forms having a focal point at or within the targeted treatment site.

Due to the wide range of beneficial treatments available it is believed preferable that the optimal use of one or more wave generators or sources should be selected on the basis of the specific application. Wherein relatively small target sites may involve a single wave generator placed on an adjustable manipulator arm. A key advantage of the present inventive methodology is that it is complimentary to conventional medical procedures. In the case of any operative surgical procedure the surgical area of the patient can be bombarded with these low energy waves to stimulate cellular release of healing agents and growth factors. This will dramatically reduce the healing process time. Most preferably such patients may be provided more than one such treatment with an intervening dwell time for cellular relaxation prior to secondary and tertiary post operative treatments.

The underlying principle of these shock wave therapy methods is to stimulate the body's own natural healing capability. This is accomplished by deploying shock waves to stimulate strong cells in the dermal tissue to activate a variety of responses. The acoustic shock waves transmit or trigger what appears to be a cellular communication throughout the entire anatomical structure, this activates a generalized cellular response at the treatment site, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced but also the number of applied shock wave impulses can be lowered from several thousand to as few as one or more pulses and still yield a beneficial stimulating response.

The use of shock waves as described above appears to involve factors such as thermal heating, light emission, electromagnetic field exposure, chemical releases in the cells as well as a microbiological response within the cells. Which combination of these factors plays a role in stimulating healing is not yet resolved. However, there appears to be a commonality in the fact that growth factors are released which applicants find indicative that otherwise dormant cells within the tissue appear to be activated which leads to the remarkable ability of the dermal tissue to generate new growth or to regenerate weakened vascular networks in for example the skin. This finding leads to a complimentary use of shock wave therapy in combination with stem cell therapies that effectively activate or trigger stem cells to more rapidly replicate enhancing the ability to harvest and culture more viable cells from the placenta, a nutrient culture of said stem cells, or other sources. The ability to stimulate stem cells can occur within the patient's own body activating the naturally occurring stem cells or stem cells that have been introduced to the patient as part of a treatment beneficially utilizing stem cells. This is a significant clinical value in its own right and is critical in attempts to overcome conditions of sagging skin.

The use of shock wave therapy requires a fundamental understanding of focused and unfocused shock waves, coupled with a more accurate biological or molecular model.

Focused shock waves are focused using ellipsoidal reflectors in electromechanical sources from a cylindrical surface or by the use of concave or convex lenses. Piezoelectric sources often use spherical surfaces to emit acoustic pressure waves which are self focused and have also been used in spherical electromagnetic devices.

The biological model proposed by Wolfgang Schaden provides a whole array of clinically significant uses of shock wave therapy.

Accepting the biological model as promoted by W. Schaden, the peak pressure and the energy density of the shock waves can be lowered dramatically. Activation of the body's healing mechanisms will be seen by in growth of new blood vessels and the release of growth factors.

The biological model motivated the design of sources with low pressure amplitudes and energy densities. First: spherical waves generated between two tips of an electrode; and second: nearly even waves generated by generalized parabolic reflectors. Third: divergent shock front characteristics are generated by an ellipsoid behind F2. Unfocused sources are preferably designed for extended two dimensional areas/volumes like skin. The unfocused sources can provide a divergent wave pattern a planar or a nearly planar wave pattern and can be used in isolation or in combination with focused wave patterns yielding to an improved therapeutic treatment capability that is non-invasive with few if any disadvantageous contraindications. Alternatively, a focused wave emitting treatment may be used wherein the focal point extends preferably beyond the target treatment site, potentially external to the patient. This results in the reduction of or elimination of a localized intensity zone with associated noticeable pain effect while providing a wide or enlarged treatment volume at a variety of depths more closely associated with high energy focused wave treatment. The utilization of a diffuser type lens or a shifted far-sighted focal point for the ellipsoidal reflector enables the spreading of the wave energy to effectively create a convergent but off target focal point. This insures less tissue trauma while insuring cellular stimulation to enhance the healing process and to effectively remodel the dermal tissue of the patient.

This method of treatment has the steps of, locating a treatment site, generating either convergent diffused or far-sighted focused shock waves or unfocused shock waves, of directing these shock waves to the treatment site; and applying a sufficient number of these shock waves to induce activation of one or more growth factors thereby inducing or accelerating healing and tissue and organ remodeling or repair.

The unfocused shock waves can be of a divergent wave pattern, planar or near planar pattern preferably of a low peak pressure amplitude and density. Typically, the energy density values range as low as 0.000001 mJ/mm² and having a high end energy density of below 1.0 mJ/mm², preferably 0.20 mJ/mm² or less. The peak pressure amplitude of the positive part of the cycle should be above 1.0 and its duration is below 1-3 microseconds.

The treatment depth can vary from the surface to the full depth of the treated tissue. The treatment site can be defined by a much larger treatment area than the 0.10-3.0 cm² commonly produced by focused waves. The above methodology is particularly well suited for surface as well as sub-surface soft tissue organ treatments as is found in the dermal regions.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. An improved method of treating excess, sagging skin of a patient using acoustic shock waves comprises the steps of:
providing a patient with excess sagging skin tissue in need of an acoustic shock wave treatment;
applying a liquid based topical treatment to surfaces of the excess sagging skin and an acoustic shock wave applicator to acoustically couple skin surfaces to enhance transmission of the acoustic shock waves;
placing an acoustic shock wave applicator on a surface of the skin tissue;
activating an acoustic shock wave generator or source to emit acoustic shock waves from an acoustic shock wave applicator; and
wherein the acoustic shock waves are transmitted from the acoustic shock wave applicator through the surface sending the emitted acoustic shock waves into the skin tissue of the patient by holding or pressing the applicator firmly against skin surfaces to enhance the acoustic coupling and to drive a portion of the topical treatment below the skin surface into the dermis tissue.

2. The improved method of claim 1, wherein the step of activating the acoustic shock wave generator or source emits low energy or unfocused acoustic shock waves.

3. The improved method of claim 1, wherein the acoustic shock waves are waves having amplitudes above 0.1 MPa and rise times of the amplitude are below 100 nano-seconds with a duration of a shock wave being below 3 micro-seconds for the positive part of a cycle and wherein the pressure pulses are an acoustic pulse which includes several cycles of positive and negative pressure with amplitudes of the positive part of such a cycle being above 0.1 MPa and the pressure pulse time duration is from below a microsecond to about a second, rise times of the positive part of the first pressure cycle is in the range of nano-seconds up to several milli-seconds.

4. The improved method of claim 1, further includes subjecting the skin to the acoustic shock waves stimulating said excess sagging skin, the skin being positioned within a path of the emitted shock waves stimulating a cellular response.

5. The improved method of claim 1, wherein the acoustic shock wave applicator is electrohydraulic and has a fluid filled flexible membrane.

6. The improved method of claim 1, wherein the acoustic shock wave applicator is either a ballistic, a piezoelectric, a radial or a spherical applicator device.

7. The improved method of claim 1, wherein the emitted shock waves or pressure pulses are convergent, divergent, planar or near planar.

8. The improved method of claim 7, wherein the emitted shock waves or pressure pulses are convergent having one or more geometric focal volumes or points located at a distance X, X being defined as the distance from an exit window to the one or more focal volumes or points from the generator or source, the excess sagging skin being positioned at the distance X or less than the distance X from the exit window source.

9. The improved method of claim 1, wherein the method of treatment further has the step of increasing the temperature of the skin being treated to change tissue impedance to improve tissue stimulation by opening pores of the skin to absorb the topical treatment deep into the dermis below the surface as the acoustic shock waves impinge the skin.

10. The improved method of claim 1, wherein the topical based treatment is a composition having collagen as an active ingredient.

11. The improved method of claim 10, wherein the composition contains one or more of glycerin, elastin, live yeast cell derivative, hyaluronic acid, preservatives, protein fractions, peptides or other suitable skin treatment components.
